(19) United States
(12) Patent Application Publication
(10) Pub. No.: US 2023/0039042 A1
(43) Pub. Date: Feb. 9, 2023

(54) MUSCLE ACTIVATION, AND ASSOCIATED ALGORITHMS, SYSTEMS AND METHODS

(71) Applicant: Strive Tech Inc., Bothell, WA (US)

(72) Inventors: Nikola Mrvaljevic, Bothell, WA (US); Zach Shelly, Bothell, WA (US); Quinn Abrahams-Vaughn, Bothell, WA (US)

(21) Appl. No.: 17/881,974

(22) Filed: Aug. 5, 2022

Related U.S. Application Data
(60) Provisional application No. 63/230,638, filed on Aug. 6, 2021.

(51) Int. Cl. A61B 5/22; A61B 5/0205; A61B 5/389; A61B 5/00

(52) U.S. Cl. CPC A61B 5/222; A61B 5/0205; A61B 5/389; A61B 5/742; A61B 5/746; A61B 5/7405; A61B 5/6801; A61B 5/024

(57) ABSTRACT

Systems and methods for tracking performance are provided. A method includes monitoring a first amplitude of a first muscle activity for a plurality of time. The method includes generating a plurality of measures of neuromuscular work for the plurality of time points using the first amplitude. The method includes determining a plurality of muscle activation quotients for the plurality of time points, using the plurality of measures of neuromuscular work. The method includes defining a plurality of muscle activation zones using the plurality of muscle activation quotients, wherein individual muscle activation zones correspond to ranges of values of the plurality of muscle activation quotients. The method includes generating a visualization of the plurality of muscle activation quotients and the plurality of muscle activation zones for the plurality of time-points. The method also includes outputting the visualization to a display.

### CROSS-REFERENCE TO RELATED APPLICATIONS

This application claims benefit of provisional application number U.S. 63/230,638 filed on Aug. 6, 2021, the contents of which are incorporated herein by reference.

### BACKGROUND

It is well known that athletes, whether professional or otherwise, are subject to injuries resulting from over-exertion, improper training, or insufficient training or conditioning. In some cases, such injuries are preceded with signs of fatigue. When a trainer (e.g., an athletic trainer or coach) detects signs of fatigue, the trainer can intervene to reduce the likelihood of fatigue-related injury. For example, when a trainer detects fatigue, the trainer may instruct the athlete to slow down or focus on technique or the trainer may pull the athlete from a game or a practice session for rest and recovery. Additionally or alternatively, the trainer may provide recommended exercise that is typically less strenuous than the normal exercise. Such observation, assessment, and intervention are typically provided based on the trainer's intimate long-term knowledge about a specific athlete and intuition built on years of experience and training.

Measurement of activity during periods of physical exertion may include measurement of heart rate by heart rate monitors, for example, as wrist-borne electronic peripherals. Heart rate alone, however, does not provide insight into cardiorespiratory or neuromuscular fatigue, which are the two main types of fatigue experienced by athletes during activity. Instead, heart rate is typically used as a heuristic to guide the athlete, for example, against benchmarks for endurance or training intensity that are reinforced by expert feedback from a trainer.

There currently exists no system or method for determination of neuromuscular work done by an athlete during a period of exertion. Similarly, quantification or algorithmic determination of neuromuscular fatigue, a useful indicator of training efficacy and injury risk, is currently unavailable.

### BRIEF DESCRIPTION OF THE DRAWINGS

The foregoing aspects and many of the attendant advantages of this invention will become more readily appreciated with reference to the following detailed description, when taken in conjunction with the accompanying drawings, where:

FIG. 1 is a diagram illustrating an analytics system configured in accordance with an embodiment of the present technology.

FIG. 2 is a diagram illustrating components of a system in accordance with an embodiment of the presently disclosed technology.

FIG. 3 is block diagram illustrating components of a system in accordance with an embodiment of the presently disclosed technology.

FIG. 4A is a schematic diagram showing a front-view of a measurement system in accordance with embodiments of the presently disclosed technology.

FIG. 4B is a schematic diagram showing a back-view of a measurement system in accordance with embodiments of the presently disclosed technology.

FIG. 5 is a paired graph of muscle activation and heart rate versus time for endurance exercise in accordance with embodiments of the presently disclosed technology.

FIG. 6 is a paired graph of muscle activation and heart rate versus time for high intensity exercise in accordance with embodiments of the presently disclosed technology.

FIG. 7 is a paired graph of work versus time, compared to a calibration curve in accordance with embodiments of the presently disclosed technology.

FIG. 8 is a flowchart illustrating an example method for measuring muscle activation and determining cardiorespiratory and neuromuscular fatigue in accordance with embodiments of the presently disclosed technology.

### DETAILED DESCRIPTION

Inventive technology is directed to determining neuromuscular work done by an athlete during a period of exertion, also encompassing quantification or algorithmic determination of neuromuscular fatigue. In the context of this application, the term athlete encompasses professional and amateur athletes, as well as hobbyists, people who exercise, either regular or irregular basis, and others who engage in sports or exercise. All such categories of people (professional, amateur, consumers, etc.) are referred to as “athletes” in this application for simplicity and brevity.

In some embodiments, the athlete's uniform or other exercise clothing may be equipped with suitable sensors and/or data acquisition controllers that collect and interpret muscle activity data (e.g., muscle amplitude and frequency, heart rate, etc.). Such sensors may measure electrical impulses of the muscles representing muscle activity data. Collected data may be algorithmically processed to indicate muscle amplitude and/or frequency for one or more muscle groups of the user. In some embodiments, the algorithmic processing may include artificial intelligence and/or machine learning.

In some embodiments, determination of neuromuscular work done by an athlete during a period of exertion may be based on data collected using a wearable sensor platform incorporating the sensors and/or data acquisition controllers. Similarly, quantification or algorithmic determination of neuromuscular fatigue and/or cardiovascular fatigue, a useful indicator of training efficacy and injury risk, may be provided by computer systems (e.g., servers, client computing devices, and/or edge devices) in communication with the wearable sensor platform. Algorithmic approaches may include rules-based procedural models, heuristic models, object models, or machine-learning models, developed for the athlete and/or the activity being monitored.

Collectively, neuromuscular and cardiorespiratory monitoring may provide improved quantitative and/or qualitative training and assessment of performance, attainment of training targets, and/or fatigue assessment. In many embodiments, monitoring of neuromuscular work, as well as cardiorespiratory work, may protect an athlete from fatigue or injury, while being significantly more cost effective than conventional methods where the athlete is repeatedly evaluated by an expert, such as a trainer, physical therapist, or nutritionist.

System Overview

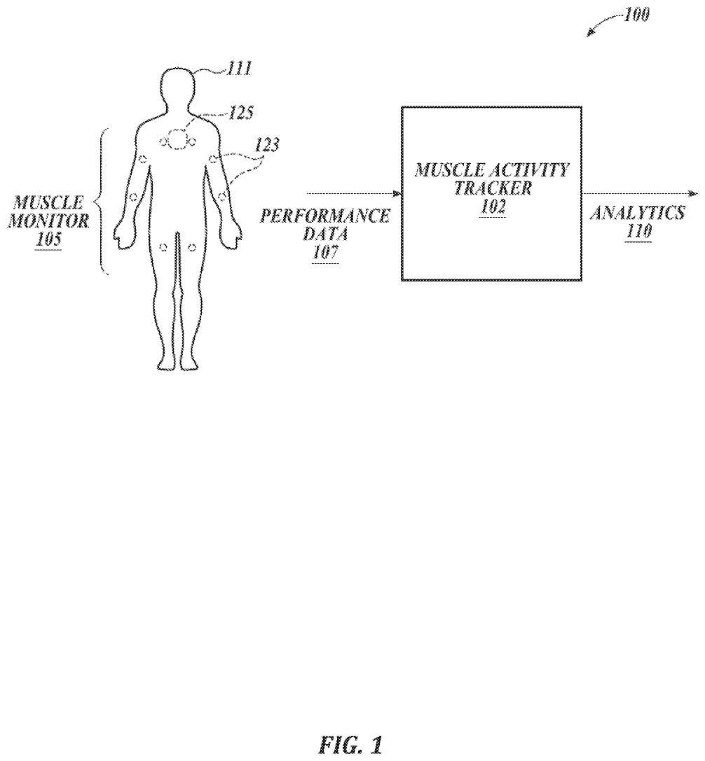

FIG. 1 is a schematic diagram illustrating an example analytics system 100 configured in accordance with an embodiment of the present technology. The system 100 includes a muscle activity tracker sub-system 102 (“muscle activity tracker 102”) and a muscle monitoring sub-system 105 (“muscle monitor 105”) that is worn by a user, such as an athlete or a user 111. The muscle monitor 105 may include an on-board controller 125 (“controller 125”) and sensors 123 that can be integrated into the athlete's clothing (not shown), such as the athlete's shirt, pants, shoes, etc. The athlete's clothing and the integrated controller 125 and sensors 123 may be collectively referred to as “smart compression clothing.” In operation, the controller 125 is configured to produce real-time or near real-time performance data (“real-time data”) 107 during an exercise, live game, practice session, or conditioning. Analytics 110 may include muscle response (MR) data, like frequency and amplitude activity for different groups of muscles, as well as motion information, as may be collected by a wearable accelerometer borne by the athlete. In different embodiments, analytics 110 may include data related to orientation state (OS) of the user, acceleration of the user, activity state (AS) of the user, etc. The analytics 110 may be produced over an evaluation period of a certain duration (e.g., 1 hour, 30 minutes, 15 minutes, 5 minutes, etc.). As described below, the system 100 can use the analytics 110 to produce indications, warnings, and alarms that alert the user or the trainer when an athlete is fatigued or injured. The system 100 can also produce indications of whether athlete's posturing, running, walking, etc. is appropriate for a given activity, and/or whether the athlete is using proper equipment (e.g., shoes, uniform, exercise weights, etc.).

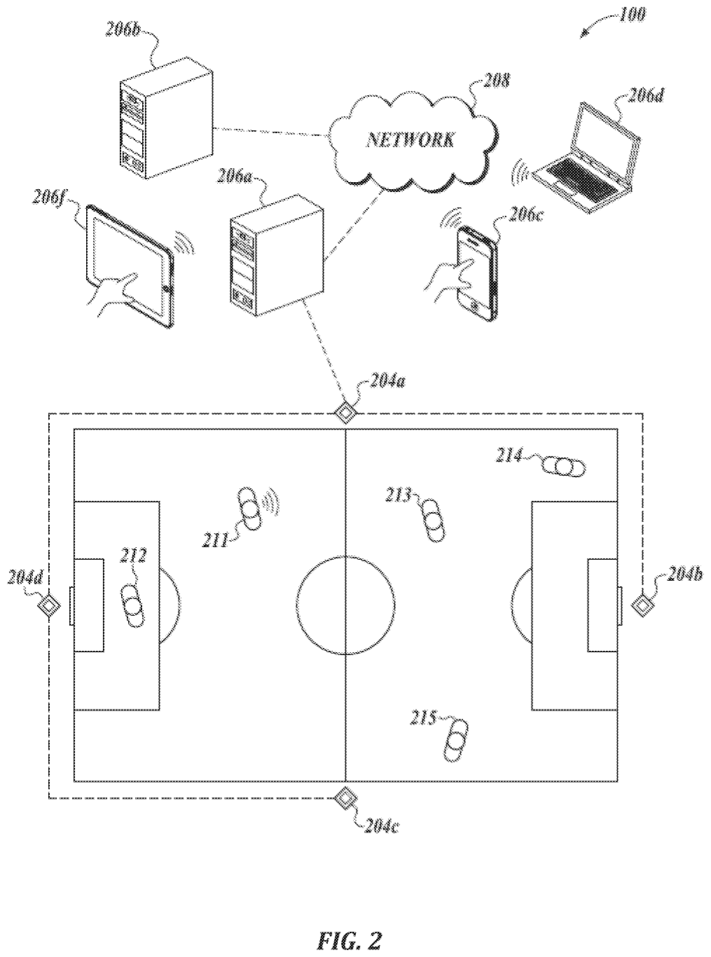

FIG. 2 is a schematic diagram illustrating components of an example analytics system 100 in further detail, in accordance with various embodiments. The system 100 illustrates interactions with multiple athletes, however, in other embodiments, the system may be focused on a single athlete. Furthermore, in different embodiments, the system 100 may include a subset of the illustrated components or additional components to those that are illustrated.

The muscle monitor 105 shown in FIG. 1 may be configured to communicate with one or more computing devices 206 via a plurality of gateway devices 204 positioned along monitoring region 227, such as a soccer-field, an athletic arena, gym, etc. The computing devices 206 are connected to one another via a network 208. The computing devices 206 are configured to receive, view, evaluate, store, and/or otherwise interact with data associated with the analytics 110 (FIG. 1). For example, intermediary or back-end server devices 206a and 206b can exchange and process communications over the network 208, store a central copy of data, globally update content, etc. Examples of well-known computing devices, systems, environments, and/or configurations that may be suitable for use with the technology include, but are not limited to, personal computers, server computers, handheld or laptop devices, cellular telephones, tablet devices, multiprocessor systems, microprocessor-based systems, set-top boxes, programmable consumer electronics, network PCs, minicomputers, mainframe computers, databases, distributed computing environments that include any of the above systems or devices, or the like.

One or more computing devices 206 can be configured to individually or collectively carry out the functions of the performance tracker 102 (FIG. 1) for producing the analytics 110. In various embodiments, the various computing devices 206 can process real-time data produced by one or more athletes 211-215 that are monitored in the monitoring region 227 of the gateways 204. As described below, the gateways 204 are configured to forward the real-time data 107 (FIG. 1) to the upstream computing devices 206 for processing.

Computing Devices

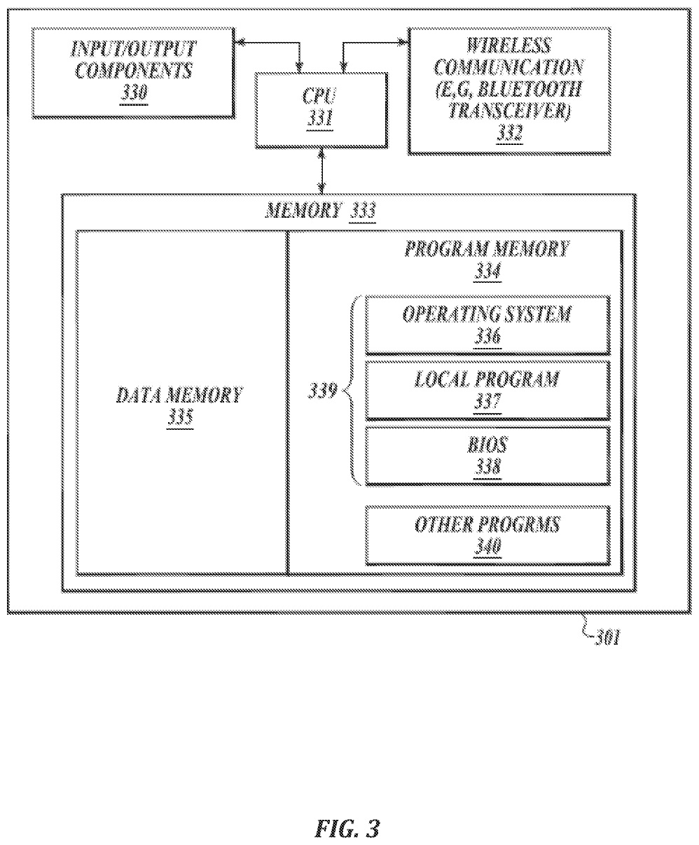

FIG. 3 is block diagram illustrating components that can be incorporated into a computing device 301, such as one of the computing devices 206 (FIG. 3), the gateways 204 (FIG. 3), and the muscle monitor 105 (1A). The computing device 301 includes input and output components 330. Input components can be configured to provide input to a processor such as CPU 331, notifying it of actions. The actions are typically mediated by a hardware controller that communicates according to one or more communication protocols. The input components 330 can include, for example, a mouse, a keyboard, a touchscreen, an infrared sensor, a touchpad, a pointer device, a camera- or image-based input device, a pointer, and/or a microphone.

The CPU 331 can be a single processing unit or multiple processing units in a device or distributed across multiple devices. The CPU 331 can be coupled to other hardware components via, e.g., a bus, such as a PCI bus or SCSI bus. Other hardware components can include communication components 332, such as a wireless transceiver (e.g., a WiFi or Bluetooth transceiver) and/or a network card. Such communication components 332 can enable communication over wired or wireless (e.g., point-to point) connections with other devices. A network card can enable the computing device 301 to communicate over the network 208 (FIG. 3) using, e.g., TCP/IP protocols. Additional hardware components may include other input/output components, including a display, a video card, audio card, USB, firewire, or other external components or devices, such as a camera, printer, thumb drive, disk drive, Blu-Ray device, and/or speakers.

The CPU 331 can have access to a memory 333. The memory 333 includes volatile and non-volatile components which may be writable or read-only. For example, the memory can comprise CPU registers, random access memory (RAM), read-only memory (ROM), and writable non-volatile memory, such as flash memory, hard drives, floppy disks, CDs, DVDs, magnetic storage devices, tape drives, device buffers, and so forth. The memory 333 stores programs and software in programming memory 334 and associated data (e.g., configuration data, settings, user options or preferences, etc.) in data memory 335. The programming memory 334 contains an operating system 336, local programs 337, and a basic input output system (BIOS) 338, all of which can be referred to collectively as general software 339. The operating system can include, for example, Microsoft Windows™, Apple iOS, Apple OS X, Linux, Android, and the like. The programming memory 334 also contains other programs and software 340 configured to perform various operations. The various programs and software can be configured to process the real-time data 107 of the athlete 111 (FIG. 2) and produce corresponding analytics, such as during the live session S1, as described in greater detail below. Those skilled in the art will appreciate that the components illustrated in the diagrams described above, and in each of the diagrams discussed below, may be altered in a variety of ways.

Clothing and Sensors

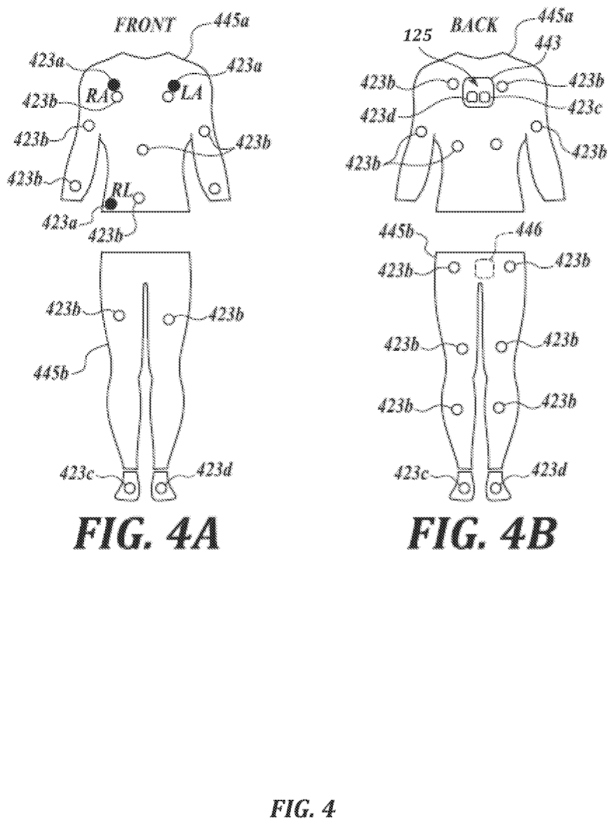

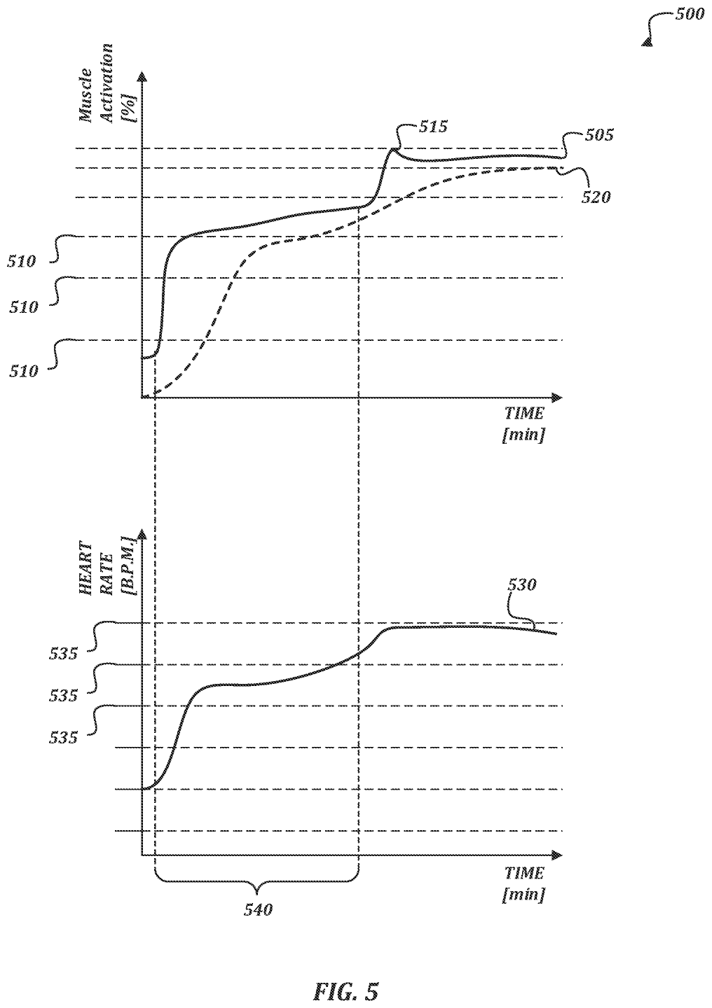

FIG. 4A and FIG. 4B are diagrams showing a measurement system in accordance with various embodiments. Referring to FIG. 5, the controller 125 can be embedded within the athlete's clothing, such as a shirt 445a and pants 445b (collectively “clothing 445”). In other embodiments, the controller 125 can be inserted into a pocket 443 in the user's clothing and/or attached using Velcro, snap, snap-fit buttons, zippers, etc. In some embodiments, the controller 125 can be removable from the clothing 445, such as for charging the controller 125. In other embodiments, the controller 125 can be permanently installed in the athlete's clothing 445.

Referring to FIG. 4A and FIG. 4B together, the controller 125 is operably coupled to muscle response sensors 423b that may be distributed over different muscle groups (e.g., pectoralis major, rectus abdominis, quadriceps femoris, biceps, triceps, deltoids, gastrocnemius, hamstring, and latissimus dorsi). The muscle response sensors 423b provide a measurement of the muscle activity during exercise. Amplitude and frequency of user's muscle response may be forwarded to the controller 125, and further to the computing devices 206 for data processing and display. A non-limiting example of the muscle response sensors 423b is an electromyography (EMG) sensor. The EMG sensors 423b can also be coupled to floating ground near the athlete's waist or hip.

In some embodiments, the clothing 445 may also be equipped with electrocardiogram (ECG) sensors 423a, orientation sensors 423c (e.g., a gyroscope), and acceleration sensors 423d (e.g., an accelerometer). Orientation sensors 423c and/or acceleration sensors 423d may be carried by the athlete's feet, for example, by being integrated and/or attached to the shoes of the athlete. The sensors 423 can be connected to the controller 125 using thin, resilient flexible wires (not shown) and/or conductive thread (not shown) woven into the clothing 445. The gauge of the wire or thread can be selected to optimize signal integrity and/or reduce electrical impedance.

The sensors 423a and 423b can include dry-surface electrodes distributed throughout the athlete's clothing 445 and positioned to make skin contact beneath the clothing along predetermined locations of the body. The fit of the clothing can be selected to be sufficiently tight to provide continuous skin contact with the individual sensors, allowing for accurate readings, while still maintaining a high-level of comfort, comparable to that of traditional compression fit shirts, pants, and similar clothing. In various embodiments, the clothing 445 can be made from compressive fit materials, such as polyester and other materials (e.g., Elastaine) for increased comfort and functionality. In some embodiments, the controller 125 and the sensors 423 can have sufficient durability and water-resistance so that they can be washed with the clothing 445 in a washing machine without causing damage. In these and other embodiments, the presence of the controller 125 and/or the sensors 423 within the clothing 445 may be virtually unnoticeable to the athlete. In one aspect of the technology, the sensors 423 can be positioned on the athlete's body without the use of tight and awkward fitting sensor bands. In the context of this application, the sensors 423 and the controller 125 are referred to as “wearable” components. In general, traditional sensor bands are typically uncomfortable for an athlete, and athletes can be reluctant to wear them.

In additional or alternate embodiments, the muscle monitor 105 (FIG. 2) can include a separate controller 446 worn on the athlete's pants 445b. The separate controller 446 can be similar to the controller 125 worn on the athlete's shirt 445a, and is connected to the individual sensors 423 located on the pants 445b. The separate controller 446 can be configured to communicate with the controller 125 and/or with the gateways 204 (FIG. 3).

Controller Communication

In operation, the controller 125 of the muscle monitor 105 is configured to process and packetize the data it receives from the sensors 423 (e.g., the muscle response sensors 423b). The controller 125 may broadcast the packetized data for detection by the gateway devices 204, which, in turn, forward the data to the muscle monitor 105 (1A) to produce analytics (e.g., frequency and amplitude of muscle activity).

Muscle Activity Indication

FIGS. 5-7 are paired graphs of neuromuscular activation and cardiorespiratory activation versus time collected over a period of time corresponding to a period of physical exertion, in accordance with embodiments of the presently disclosed technology. In each graph, the horizontal axis represents time and the vertical axis represents derived quantities including, but not limited to, muscle activation quotient, heart rate, work, or deviation from nominal performance. The illustrated graphs represent time series of the amplitude activity for muscle groups, heart rate data, and/or motion data, measured continuously or intermittently, for example, by sampling muscle response sensors 423b of FIG. 4 for multiple time points during the period of physical exertion. The graphs are intended to be illustrative of dynamic visualizations presented on a display, as described in more detail in reference to FIGS. 2-3, as part of real time or near-real time training, treatment, or observation of physical activity. The curves illustrated in FIGS. 5-7 are provided as illustrative examples and are not intended to represent real data collected for a particular athlete or to be limiting as to the performance of the systems described in reference to FIGS. 1-4B.

FIG. 5 is a paired graph 500 of muscle activation and heart rate versus time for endurance exercise in accordance with embodiments of the presently disclosed technology. The paired graph 500 is an illustrative example of data that may be generated by monitoring muscle activity during a cardio-workout or other endurance exercise or activity, using an amplitude sensor over a period of time at least partially overlapping a period of physical exertion. As a derived quantity, the systems described in reference to FIGS. 1-4B may generate measures of neuromuscular work as a function of time, using the amplitude data for one or more muscle groups. For example, a measure of neuromuscular work may be a measurement from which a real-time or near-real time muscle activation curve 505 may be derived.

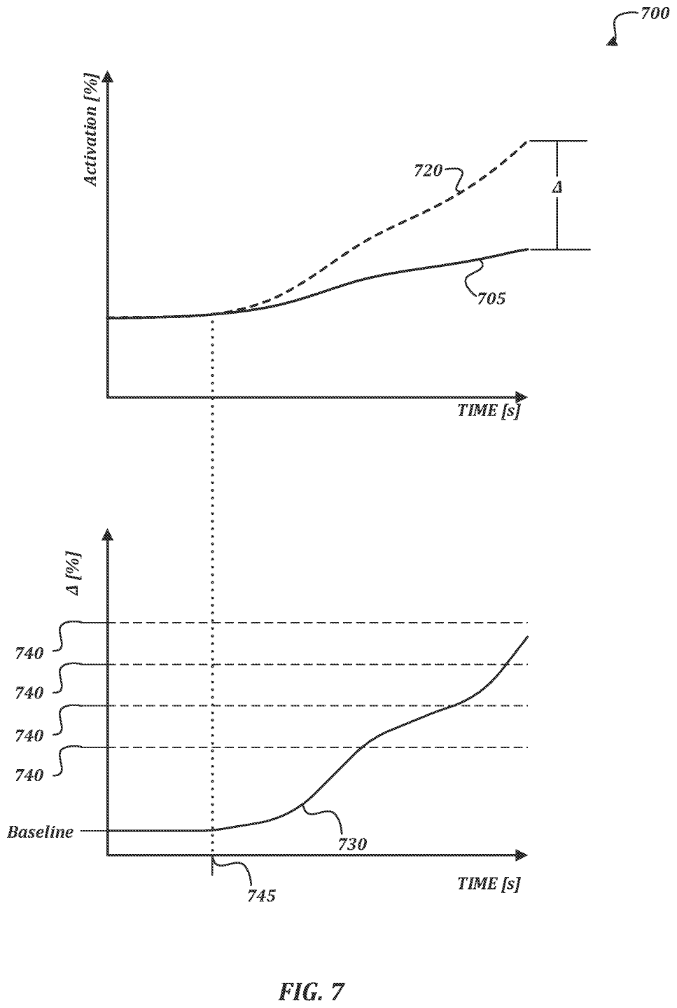

Neuromuscular work and a muscle activation quotient may be determined using muscle amplitude measurements by several techniques including, but not limited to, rules-based models, object models, or by machine learning or other predictive techniques. In this way, direct monitoring of muscle activity may be correlated to the muscle activation quotient, for example, as a percentage value of a maximum muscle amplitude. In an illustrative example, longitudinal data aggregation over multiple exercise or activity sessions, such as during a training camp or over a course or regimen, may permit generating multiple characteristic parameters for an athlete including, but not limited to, muscle group-specific activation windows, temporal characteristics from which the type of activity may be estimated automatically (e.g., without explicit human identification of the type of activity), or activity patterns that can be used to derive calibration data, as described in more detail in reference to FIG. 7.

In some embodiments, one or more parameters of a rules-based algorithm, such as a heuristic model, are developed by longitudinal data collection of an athlete during a specific exercise. For example, for the endurance exercise illustrated in paired graph 500, activation curve 505 may be derived by inputting muscle activity measurements for multiple time points into a heuristic model developed for the athlete. In this way, the muscle activation may be determined as a percentage of a nominal, maximum, average, target, or other activity value.

Analogously, object models or machine-learning models may be implemented as part of the algorithms for determining neuromuscular work and muscle activation, as an approach to account for hidden parameters or interactions between parameters of training sessions that may be difficult to identify and capture in deterministic models. For example, confounding factors including, but not limited to, time of day, altitude, frequency of training or activity sessions, donning or doffing of weights, team composition, physiological cycles, or other factors, may interact to result in fatigue or injury in ways that are difficult to predict analytically. As an illustrative example, longitudinal muscle activity data for a variety of training conditions may be combined with environmental and other metadata to constitute a training set for an artificial neural network model, which may be used to train the neural network model to predict the muscle activation quotient in a way that accounts for the role of atmospheric pressure, humidity, temperature, running surface, or other factors.

Advantageously, determining neuromuscular work, also referred to as muscle load, can permit multiple types of activities to be compared quantitatively. For example, activities can be classified based at least in part on whether physical exertion moves the athlete (free-movement type) or whether physical exertion moves an object relative to the athlete (relative-movement type). Examples include running and lifting, respectively. For activities where the athlete moves, motion data can be used to generate an external load parameter, measuring the work done to move the athlete, which in turn can be used to compare between different activities of the same type. External load, in this context refers to parameters based at least in part on motion data that provide a dimensionless metric of physic exertion used to generate motion of the athlete (e.g., rate of change of acceleration, total acceleration over a predefined distance, etc.) For relative-movement exercises, such as lifting, motion of the wearer is limited, and the same external load parameter is less meaningful. To that end, one or more statistical models can be used to generate muscle load data, developed by fitting muscle data to external load data as an approach to generating a neuromuscular work value that can be used to compare different types of activities for which external load may be unavailable or less meaningful.

In some embodiments, statistical models for generating muscle load data can include regression models developed from combined motion and muscle activity data for multiple athletes. By fitting the regression models to point data, using a linear trend line defined by equating muscle load with external load (e.g., muscle load=external load) as a goal, motion data can be used to fit models that permit muscle activity sensor data to be used to generate measures of neuromuscular work. Similarly, muscle activity data can be used to determine external load. One fitted, muscle load models can be used to generate a dedimensionalized parameter of neuromuscular work, as part of training, fatigue monitoring, and athletic performance assessment.

In an illustrative example, the regression model can include a square root of the sum of six EMG sensor signals, provided that the sum can be modified with model coefficients developed during regression fitting. In another example, the regression model can include an average (e.g., mean) of six EMG sensor signals, provided that the average can be modified with model coefficients developed during regression fitting. In another example, the regression model can include a square root of the average (e.g., mean) of six EMG sensor signals, provided that the average can be modified with model coefficients developed during regression fitting. In another example, the regression model can include model coefficients that are a function of the average (e.g., mean) of six EMG sensor signals. While these illustrative examples include six sensors, the number of sensors can be greater or fewer, depending on the muscle(s) being measured, the type of activity, or the size of the wearer.

In some embodiments, qualitative and/or quantitative information may be derived from the muscle activation quotient by defining multiple muscle activation zones 510. For example, individual muscle activation zones 510 may correspond to contiguous ranges of values of the muscle activation quotient and may be defined in one or more ways relative to the muscle activation quotient. In some cases, defining the muscle activation zones 510 includes estimating a maximum muscle activation quotient 515 using the real time muscle activation curve 505 and defining the muscle activation zones 510 in relation to the maximum muscle activation quotient 515. While the maximum muscle activation quotient 515 is illustrated as being included in the real time muscle activation curve 505, in some embodiments, the maximum muscle activation quotient 515 may reach a fraction of a maximum value that is determined by longitudinal data collection. In this way, the muscle activation zones 510 may be defined and/or refined over multiple activity sessions. In some embodiments, the maximum muscle activation quotient 515 may reach about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, of the maximum value that is determined by longitudinal data collection. In some embodiments, the maximum muscle activation quotient 515 may be defined as part of training or recovery activity.

In some embodiments, muscle activity data may be collected at a sampling rate that is optimized to conserve one or more resources of the wearable sensor system. For example, in an endurance activity lasting several hours, the sensors may be active intermittently, such that the maximum muscle activation quotient 515 may occur between sampling points. In this way, estimating the maximum muscle activation quotient 515 may include interpolating one or more estimated muscle activation quotient datapoints between the sampling time points and identifying the maximum muscle activation quotient from the interpolated muscle activation quotient datapoints. Such approaches may provide improved performance and suitability of wearable sensor platforms for assessment of both cardiorespiratory and neuromuscular work and fatigue, for a broad range of activities, including activities that take an athlete away from sources of electricity or network connectivity.

The muscle activation zones 510 may be defined as a linear proportion of the maximum muscle activation quotient. Higher-order dynamics of physiological systems may be reflected by defining the muscle activation zones 510 by a natural-logarithmic proportion of the maximum muscle activation quotient. Similarly, a logarithmic proportion, a sigmoidal proportion, a parabolic proportion, or other converging functional proportion may be used to define the muscle activation zones 510.

Advantageously, defining muscle activation zones 510, such as those for which the range decreases as the muscle activation quotient increases, may permit discretization of physical exertion into one or more muscle activation zones as an approach to targeting particular levels of neuromuscular work. Such techniques may benefit the athlete, for example, by avoiding overwork that may result in muscle degradation, lactic acid buildup, or acute injury or strain. In this way, neuromuscular work may be managed in a manner akin to heart rate management.

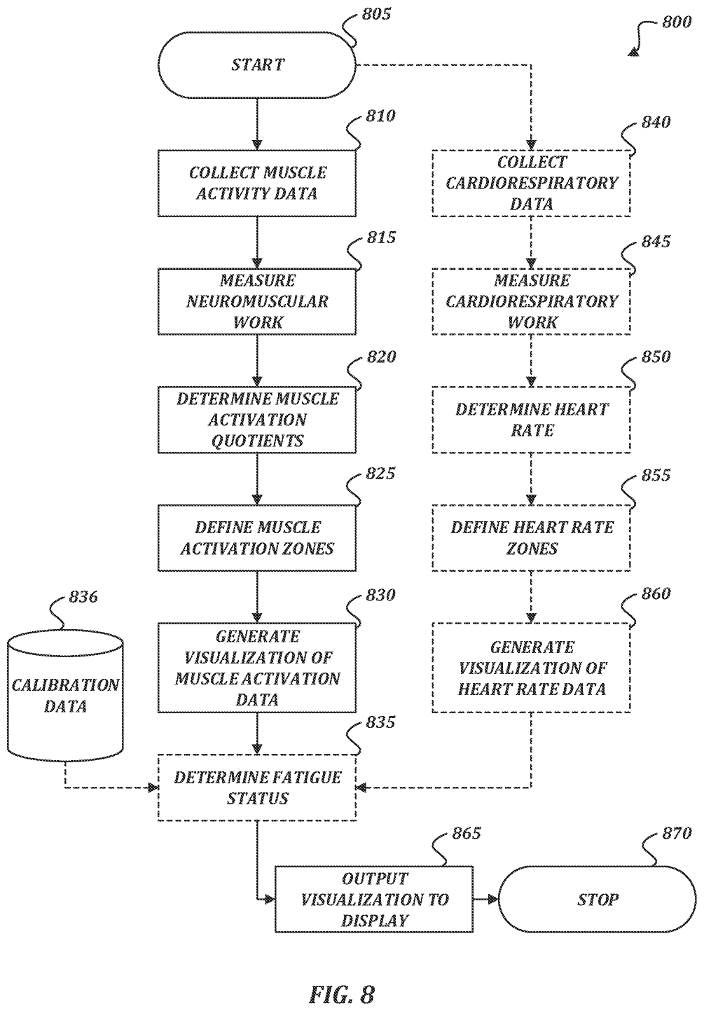

The real time muscle activation curve 505 may be time-averaged over a dynamic averaging window, for example, over a portion of the period of time or over the entire period of time, from which a smoothed muscle activation curve 520 may be derived. Advantageously, smoothing the real time muscle activation curve 505 may provide improved qualitative and/or quantitative assessment or prediction of neuromuscular fatigue. For example, while real-time muscle activation data may include noise propagated from electromyography sensors, as well as rapid dynamics that may induce large changes in predictive models that include derivative terms, smoothed muscle activation data may be more intuitive for viewing on a display, and may provide more meaningful output from a model, for example, as a function of time. Smoothing may be applied to sensor data directly or may also be applied to muscle activation quotient data. For smoothed muscle activation quotient data, smoothing may reduce the likelihood that system analytics (e.g., analytics 110 of FIG. 1) may generate a warning prompt that the activity is outside one of the muscle activation zones 510 that is being targeted. Warning prompts, including auditory prompts, are described in more detail in reference to FIG. 8.

In conjunction with the muscle activation quotient, activity may be monitored to determine cardiorespiratory work and to predict and/or identify cardiorespiratory fatigue by measuring heart rate as a function of time. As shown in the paired graph 500, a heart rate curve 530 may be generated by monitoring the amplitude of sensor configured for measuring the pulse. For example, the heart rate may be measured by a wearable sensor (e.g., sensors 423 of FIG. 4) that may be incorporated into clothing and/or an electronic peripheral (e.g., a smart watch). Analogous to the muscle activation zones 510, multiple heart rate zones 535 may be defined using heart rate data, where individual heart rate zones may correspond to contiguous ranges of the heart rate. In this way, analytics described in relation to analysis and/or prediction of neuromuscular work, muscle activation, and neuromuscular fatigue may also be provided for cardiorespiratory work, heart rate, and cardiorespiratory fatigue.

While cardiorespiratory work and neuromuscular work describe different anatomical systems, the muscle activation curves 505 and/or 520 and the heart rate curve 530 may exhibit similar dynamics under some conditions. For example, for cardio or other endurance exercise, fatigue and exertion may develop with similar dynamics over the course of an activity session (e.g., on the order of seconds, minutes, or hours). In an illustrative example, in a swimming session of one kilometer or more, both the muscle activation quotient and the heart rate may rise to an initial plateau over a period of time 540. This may correspond to a time during which the athlete is training within the target parameters, and both the muscle activation quotient and the heart rate are within target zones. Following the period of time 540, however, additional exertion may be observed.

Without additional information, however, the dynamics illustrated in the paired graph 500 are not necessarily indicative of fatigue. For example, the athlete may have transitioned to a higher intensity interval. In the example of the swimming session, the dynamics observed following the period of time 540 may be attributable to fatigue, or to changing stroke styles, swimming speed, or other aspects of the exertion. Inference of fatigue may depend, therefore, on reference information, such as calibration data or historical/longitudinal data provided to a model. In a simple example, were the heart rate and the muscle activation quotient to diverge, rather than track closely, a model may indicate fatigue if data indicate the dynamics are anomalous. In some embodiments, multiple periods of time 540 may be defined to assess the time spent in each muscle activation zone 510. Advantageously, measures of time spent in each muscle activation zone 510 may improve training efficacy, for example, by providing analogous impact to timed cardio training targeting specific heart rate ranges.

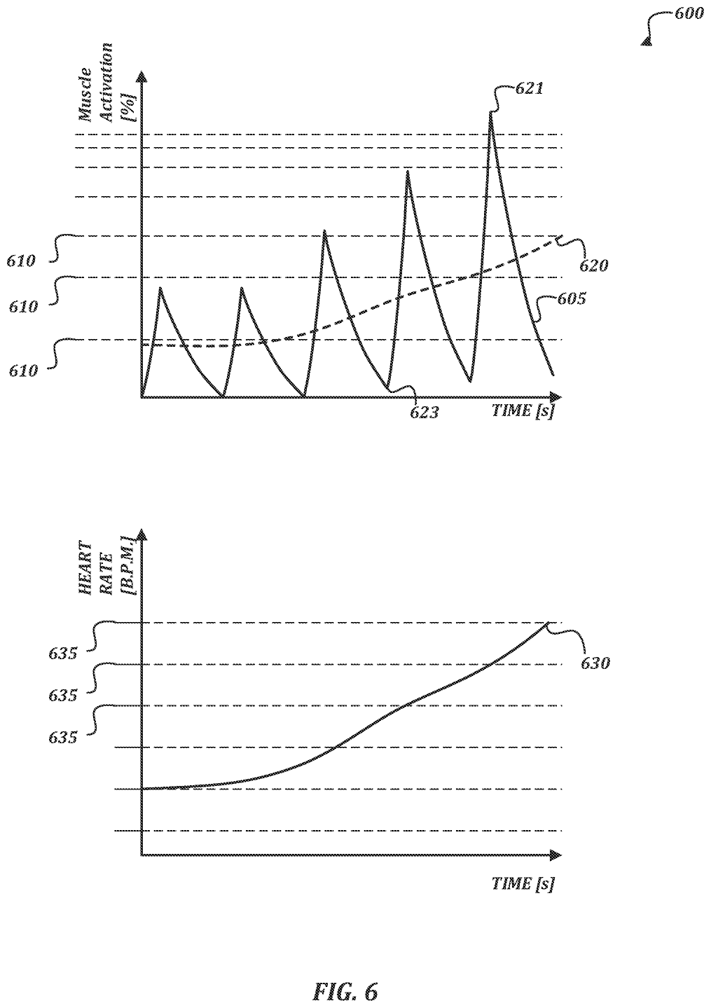

FIG. 6 is a paired graph 600 of muscle activation and heart rate versus time for high intensity exercise in accordance with embodiments of the presently disclosed technology. Examples of high intensity exercise include sprinting, weightlifting, or exercise that may target fast-twitch muscle groups and may be associated with different dynamics between neuromuscular work measurements and cardiorespiratory work measurements. For that reason, algorithms and/or processing techniques, such as smoothing or time-averaging of muscle activation quotient data, may permit improved fatigue determination, and may also provide improved qualitative and quantitative feedback to athletes to guide and enhance training. As described in reference to FIG. 5, the paired graph 600 may be presented on a display as a dynamic visualization, where the graphs may be presented on the same axis or on separate axes, for example, as part of a training feedback tool or software application hosted on a mobile electronic device.

The paired graph 600 describes, as a function of time (e.g., in seconds), a muscle activation quotient curve 605 and a time-averaged curve 620, where the muscle activation quotient is expressed in terms of fraction (e.g., a percentage value) of a comparison value including, but not limited to, a target value, a maximum value, or an average value. The muscle activation quotient, presented on the ordinate axis (e.g., y-axis), may be divided into one or more muscle activation zones 610, which may be determined as described for muscle activation zones 510 of FIG. 5. In contrast to paired graph 500 of FIG. 5, however, the muscle activation quotient curve 605 illustrates relatively greater dynamics, corresponding to repetition of a relatively high intensity muscle contraction followed by controlled relaxation of the muscle. As an illustrative example, the paired graph 600 may correspond to a leaping exercise where an athlete is repeatedly jumping in place while a wearable sensor records muscle activation in the quad, glute, or hamstring muscle groups. In another example, the paired graph 600 may correspond to a weight lifting exercise, where an athlete repeatedly lifts a weight (e.g., a bicep curl, a calf-raise, a leg lift, etc.) while wearable sensor records muscle activation of the appropriate muscle group.

Advantageously, the same or similar quantitative feedback using the muscle activation quotient curve may improve training efficacy or athletic performance, for example, by permitting the athlete to target particular activation zones 610, to identify after how many repetitions the athlete starts to overwork, to guide incrementation of weight.

While time spent in each muscle activation zone 610 may be relatively less meaningful for the muscle activation curve 605, in contrast to the muscle activation cure 505 of FIG. 5, temporal dynamics of the time-averaged curve 620 may provide meaningful quantitative insight to the athlete. For example, over the course of a repetitive high-intensity exercise, drift may be observed in the muscle activation curve 605 for both peak muscle activation 621 and baseline muscle activation 623. In this way, the time-averaged curve 620 may also describe average peak muscle activation 621, for example, by detecting peaks in the muscle activation curve 605 and averaging the peak muscle activation 621 values. Similarly, the time-averaged curve 620 may describe average peak muscle activation 621, for example, by detecting peaks in the muscle activation curve 605 and averaging the peak muscle activation 621 values. Over the course of an activity session, the time-averaged curve 620 may trend upwards, which may indicate increasing exertion and/or muscle group hysteresis.

Concurrent monitoring of cardiorespiratory work, and determination of the athlete's heartrate, may supplement the neuromuscular work measurements and may provide additional insight into training efficacy and/or athletic performance. High-intensity repetitive exercise may exhibit a time-averaged effect on heart rate, which may be attributable at least in part to the autonomic function of the heart and circulatory system. In this way, a heart rate curve 630 generated for the athlete during the course of the activity session may trend upwards as the session progresses, corresponding to increased oxygen demand at the targeted muscle groups. In this way, divergent dynamics between the time-averaged curve 620 and the heart rate curve 630 may provide similar insight into fatigue or injury, for example, where the muscle activation quotient rises acutely without corresponding changes in heart rate. Additionally or alternatively, calibration data may be collected for the athlete and for the activity, by which anomalous dynamics in either curve may be identified, and neuromuscular fatigue and/or cardiorespiratory fatigue may be determined.

FIG. 7 is a paired graph 700 of activation versus time, compared to a calibration curve in accordance with embodiments of the presently disclosed technology. The paired graph 700 describes activation as a function of time. In this context, activation may describe muscle activation quotient data or heart rate data, either of which may be referenced to calibration data and may provide insight for improving training or performance and may identify fatigue or injury before or upon onset. As described in reference to FIGS. 5-6, calibration data may be collected for an athlete and for an activity over multiple activity sessions, from which a calibration curve 705 may be generated for use in identifying anomalous information.

As an illustrative example, calibration curve 705 describes nominal time-averaged muscle activation data for an athlete during a particular high-intensity activity, such as sprinting or lifting. In this context, “nominal” describes peak or optimal performance of the monitored muscle group. In this way, the calibration curve 705 may describe a comparison standard for the athlete, as a reference for expected or “normal” performance. In comparison, a measured activation curve 720 may describe data collected during a particular activity session. While the calibration curve 705 may describe the expected or normal performance for the athlete, the measured activation curve 720 may correspond to an activity session following a competition, a series of strenuous training sessions, or an illness or injury. In this way, a differential quantity may be defined, referred to using the Greek letter delta “A,” which may be calculated as a function of time, for example, by a simple difference between the calibration curve 705 and the muscle activation curve 720. Additional or alternative techniques for determining the differential quantity may include, but are not limited to, weighting the difference as a function of time, using proportional, integrative, and/or derivative models to account for temporal dynamics in the data, inputting both the calibration data and the muscle activation data to a model to predict and/or classify fatigue and injury etc. As part of the visualizations described in reference to FIGS. 5-6, a delta curve 730 may be generated and/or presented to provide quantitative and/or qualitative information about deviation from nominal performance, for example during training or competition.

In some embodiments, the differential quantity A is used as part of an algorithm for identifying fatigue and/or predicting fatigue or injury. For example, one or more fatigue or injury zones 740 may be defined, by which the magnitude of anomalies may be determined. The injury zones 740 may be defined using algorithmic outputs that account for the same or similar meta-parameters as described above in the context of determining muscle activation zones and/or muscle activation quotient. For example, deviation from “nominal” performance may be expected in some circumstances without rising to the level of being anomalous. Such circumstances may include, but are not limited to, changes in altitude, time since most recent activity session, recover from injury, etc. In this way, a model may be integrated into an algorithm to account for meta parameters, and to more accurately define the differential quantity A and the fatigue or injury zones 740.

Similarly, dynamics in the delta curve 730 as well as amplitude of the delta curve 730 may identify respective fatigue-type (e.g., neuromuscular vs. cardiovascular) and may improve differentiation between fatigue and injury. For example, crossing from a baseline into a fatigue or injury zone 740 may indicate fatigue, while crossing into the next fatigue or injury zone 740 may indicate injury, while crossing into the next fatigue or injury zone 740 may indicate severe injury. Similarly, temporal dynamics may provide insight into the progression of an activity session from nominal performance into a fatigue or injury zone 740. For example, differential terms describing dynamics in the delta curve 730 may indicate one or more inflection or stationary points 745 that may indicate transitions from nominal performance into a fatigue onset or injury onset phase. In this way, multiple types of quantitative and/or qualitative information may be derived from differential quantity data, using longitudinal calibration data, to improve training efficacy and/or athletic performance, to predict fatigue or injury, or to guide execution of exercises or other athletic activity.

FIG. 8 is a flowchart illustrating an example method 800 for measuring muscle activation and determining cardiorespiratory and neuromuscular fatigue in accordance with embodiments of the presently disclosed technology. In some embodiments, the operations of the example method 800 may include a subset of the operations illustrated, or may include additional operations that are not illustrated in the flowchart. The individual operations of the example method 800 may be implemented by the systems described in reference to FIGS. 1-4. As such, the operations are described as part of a method implemented by a computer system. In this way, the example flow 800 may be stored as computer-executable instructions on a non-transitory computer-readable memory that, when executed by one or more processors of the computer system, may implement the operations of the flow illustrated in FIG. 8. It is understood that other systems or implementations of the example method 800 are contemplated, of which FIG. 8 describes but one example.

The example method 800 starts at block 805. At block 810, certain muscle groups may be selected for observation. Some examples of such muscle groups are right quad (RQ) and left quad (LQ), right hamstring (RH) and left hamstring (LH), etc. Selecting the much group may include electronically selecting, for example, through a user interface, of a sensor channel corresponding to one or more wearable muscle response sensors positioned relative to the selected muscle group. Positioning, as described in more detail in reference to FIG. 4A-4B, may be achieved by integrating one or more sensors into a garment or electronic peripheral.

Once selected, the muscle groups are monitored to collect muscle activity data. Muscle activity may describe nerve impulse signals generated by muscle flexion, for example, during motion of a body part or during isometric exertion. In this way, monitoring muscle activity may describe monitoring the amplitude of muscle activity of an athlete for multiple time points at least partially overlapping a period of physical exertion, such as a training session, athletic performance, or competition.

At block 815, the example method 800 includes generating one or more measures of neuromuscular work for the plurality of time points using the first amplitude. In some cases, generating the measure(s) may describe measuring the amplitude of the muscle by a first wearable muscle response sensor for multiple time points. The amplitude, in turn, may describe an electrical signal measured by an electromyography sensor positioned to monitor the muscle. The electromyography sensor may be a part of a wearable sensor system and may be an example of a wearable muscle response sensor configured for monitoring the amplitude of the muscle activity of the athlete.

Subsequent generating the measure(s) of neuromuscular work, the example method 800 may include determining one or more muscle activation quotients for the time points at block 820. As described in more detail in reference to FIG. 5, determining muscle activation quotients may include implementation of algorithms incorporating procedural models, object models, and/or machine-learning (e.g., neural network) models, that together may generate the muscle activation quotient data to quantitatively describe the extent of neuromuscular work as a function of multiple interacting factors including meta parameters, including but not limited to environmental parameters, temporal factors, physiological factors, or equipment parameters. In an illustrative example, block 820 may include inputting the muscle activity data collected at block 820 into a model including heuristics and/or an artificial neural network model for which the model is configured to also take in environmental data, such as altitude, temperature, or humidity; physiological data, such as body temperature, blood oxygen, or other data that may be quantitative, qualitative, or coded (e.g., qualitative fatigue coded on a 1-3 scale); equipment or other factors, such as gear type (shoes, skates, swimwear, etc), physical surface type, pool type, etc; or other meta-parameters that may have an impact on peak performance and endurance of an athlete. The output of the model may include a continuous curve of muscle activity quotient data for a period of physical exertion that may cover all or a part of an activity session, as described in more detail in reference to FIGS. 5-7. In some cases where operations invoke aspects of artificially intelligent systems, such as machine learning models, trained models may be optimized for execution on an edge device incorporating the display, such as an activity tracker device, while training of the models may be implemented at remote systems, such as cloud systems or on servers equipped with greater computational resources.

At block 825, the example method 800 may include defining one or more muscle activation zones for the athlete using the muscle activation quotient data generated at block 820. Individual muscle activation zones may correspond to contiguous ranges of values of the plurality of muscle activation quotients. As described in more detail in reference to FIG. 5, block 820 may include estimating a maximum muscle activation quotient using the muscle activation quotient data and defining the muscle activation zone(s) in relation to the maximum muscle activation quotient. The maximum muscle activation quotient may be contained within the data monitored as part of the example method 800, may be a longitudinally derived value over a number of activity sessions (e.g., over multiple iterations of the example method 800), or may be a user-defined method provided as part of a training or therapy regimen. In this way, the muscle activation zones may be defined in one or more approaches, including but not limited to a linear proportion of the maximum muscle activation quotient; a natural-logarithmic proportion of the maximum muscle activation quotient; or other approaches, as described in more detail in reference to FIG. 5. In some embodiments, block 824 may include estimating a period of time corresponding to a muscle activation zone, based on the time an athlete spends in a given muscle activation zone.

Subsequent defining the muscle activation zones, the example method 800 may include generating a visualization of the muscle activation quotient data and the muscle activation zones for the activity session at block 830. Generating the visualization may describe one or more operations associated with generating datasets in one or more graphical data formats, such as comma-separated value datasets or other data formats compatible with one or more visualization software applications. The visualization data may be generated for presentation on a display that is incorporated into a mobile electronic device, such as a mobile electronic device hosting software for implementing one or more operations of the example method 800.

In some embodiments, the example method 800 may optionally include determining a fatigue status at block 835. The fatigue status may describe neuromuscular fatigue, cardiorespiratory fatigue, and/or injury indication information. Determining the fatigue status may include defining a comparison between the muscle activation quotient data and a calibration curve for the athlete and/or for the activity and determining the fatigue status for the athlete using the comparison. In some embodiments, determining a fatigue status for the athlete may include defining a second comparison between heart rate data and a cardiorespiratory calibration curve for the athlete. In this way, cardiorespiratory fatigue, as well as neuromuscular fatigue, may be determined.

As described in more detail in reference to FIG. 7, the comparison may describe one or more techniques for comparing current performance against “nominal” or “normal” performance. Techniques for the comparison may include, but are not limited to, direct comparison by with calibration data, which may be stored in a database 836 storing the calibration data. The database 836 may be in electronic communication with the system performing the operations of the example method 800, for example, over a network. In this way, the database 836 may store additional data generated as part of the example method 800, such as the muscle activation quotient data, the muscle activation zone data, the heart rate data, or other data. In some embodiments, the database 836 may include multiple data storage systems communicating over a database, as in a distributed storage system.

In some embodiments, the example method may optionally include monitoring and collection of cardiorespiratory data at block 840, as part of a series of optional operations directed at generating quantitative and/or qualitative measures of cardiorespiratory work and fatigue. Blocks 840-860, therefore, may be executed concurrently or in parallel with the operations of blocks 810-830, such that real-time output of the example method 800 may include visualizations of both muscle activation data, heart rate, and fatigue/injury status on a display at block 865. Additional or alternative outputs of the example method 800 may include storing data collected or generated as part of the operations of the example method 800 in the database 836 or in other databases in communication with the computer system implementing the example method 800.

The operations of blocks 840-860, described in more detail in reference to FIGS. 5-7, may represent an improvement of typical approaches to measuring or estimating cardiorespiratory fatigue, in that improvements to modeling and holistic data collection may be implemented through collecting heart-rate data as part of a wearable sensor platform, rather than through a single electronic peripheral, such as a smart watch or heart rate monitor.

In some embodiments, determining fatigue status 835 and outputting visualization data to the display at block 865 may include operations for generating one or more prompts for a user of the system or the athlete donning the wearable sensor platform. The prompt(s) may include warning information indicative of fatigue or injury, determined, for example, when muscle activation quotient data exceeds a threshold value for safe exertion. The prompt may be present using the display, or may be provided by alternative modalities, such as an auditory prompt including the warning information, which may be generated using an acoustic speaker in electronic communication with the system (e.g., an integrated speaker in a smartphone or tablet).

While various advantages associated with some embodiments of the disclosure have been described above, other embodiments may also exhibit such advantages, and not all embodiments need necessarily exhibit such advantages to fall within the scope of the invention. For example, while various embodiments are described in the context of an athlete (e.g., a professional or collegiate athlete), in some embodiments users of the system can include novice or intermediate users, such as users, trainers, and coaches associated with a high school sports team, an athletic center, a professional gym, physical therapist, etc. Accordingly, the disclosure can encompass other embodiments not expressly shown or described herein. Unless otherwise noted, in the context of this disclosure, the word “approximately” indicates a difference of +/−5% of the stated value.

What is claimed is:

1. A method for tracking performance during a period of physical exertion by an athlete, the method comprising:
monitoring a first amplitude of a first muscle activity of an athlete for a plurality of time points that at least partially overlap with the period of physical exertion;
generating a plurality of measures of neuromuscular work for the plurality of time points using the first amplitude;
determining a plurality of muscle activation quotients for the plurality of time points, using the plurality of measures of neuromuscular work;
defining a plurality of muscle activation zones for the athlete using the plurality of muscle activation quotients, wherein individual muscle activation zones correspond to ranges of values of the plurality of muscle activation quotients;
generating a visualization of the plurality of muscle activation quotients and the plurality of muscle activation zones for the plurality of time-points; and
outputting the visualization to a display.

2. The method of claim 1, further comprising:
defining a comparison between the plurality of muscle activation quotients and a calibration curve for the athlete; and
determining a fatigue status for the athlete using the comparison.

3. The method of claim 1, wherein the visualization is a first visualization, and wherein the method further comprises:
monitoring a second amplitude of a second muscle of the athlete for the plurality of time points;
generating a plurality of measures of cardiorespiratory work for the plurality of time points;
determining a heart rate using the plurality of measures of cardiorespiratory work;
defining a plurality of heart rate zones using the heart rate, wherein individual heart rate zones correspond to contiguous ranges of the heart rate;
generating a second visualization of the heart rate and the plurality of heart rate zones for the plurality of time-points; and
outputting the second visualization to the display.

4. The method of claim 1, wherein generating the plurality of measures of neuromuscular work comprises:
monitoring a plurality of amplitudes of a plurality of muscle activities of the athlete for at least one timepoint of the plurality of timepoints; and
generating a de-dimensionalized muscle load parameter using a model configured to determine a square root of the average of the plurality of amplitudes.

5. The method of claim 4, wherein the plurality of amplitudes are monitored using one or more electromyography sensors.

6. The method of claim 1, further comprising:
estimating an average muscle activation quotient for the period of exertion using the plurality of muscle activation quotients for the plurality of time points.

7. The method of claim 1, wherein defining the plurality of muscle activation zones comprises:
estimating a maximum muscle activation quotient using the plurality of muscle activation quotients for the plurality of time points; and
defining the plurality of muscle activation zones in relation to the maximum muscle activation quotient.

8. The method of claim 7, wherein the plurality of muscle activation zones are defined by a linear proportion of the maximum muscle activation quotient.

9. The method of claim 7, wherein the plurality of muscle activation zones are defined by a natural-logarithmic proportion of the maximum muscle activation quotient.

10. The method of claim 7, further comprising:
estimating a period of time corresponding to a muscle activation zone of the plurality of muscle activation zones, using the plurality of muscle activation quotients for the plurality of time points.

11. The method of claim 7, wherein the plurality of muscle activation quotients is a first plurality of muscle activation quotients, and wherein estimating the maximum muscle activation quotient using the first plurality of muscle activation quotients for the plurality of time points comprises:
interpolating a second plurality of muscle activation quotients between the first plurality of muscle activation quotients; and
identifying the maximum muscle activation quotient from second plurality of muscle activation quotients.

12. The method of claim 1, further comprising:
determining a muscle activation quotient exceeding a threshold value for safe exertion using the plurality of muscle activation quotients; and
generating a prompt using the display, the prompt comprising warning information.

13. The method of claim 12, further comprising:
generating an auditory prompt comprising the warning information using an acoustic speaker.

14. The method of claim 1, further comprising:
storing the plurality muscle activation quotients in a data store.

15. A system for tracking performance during physical exertion by an athlete, the system comprising:
a wearable muscle response sensor configured for monitoring an amplitude of a muscle activity of the athlete;
a muscle activity tracker configured for receiving data from the muscle response sensor and for determining a plurality of muscle activation quotients as a function of time for the muscle; and
a display, electronically coupled with the muscle activity tracker and configured to present a visualization of the muscle activation quotient.

16. The system of claim 15, further comprising at least one database storing muscle activation quotient data for the athlete and configured to receive the muscle activation quotient from the muscle activity tracker.

17. The system of claim 15, wherein the system comprises one or more processors and non-transitory memory storing instructions that, when executed by the one or more processors, cause the one or more processors to perform operations comprising:
monitoring the amplitude for a plurality of time points;
generating a plurality of measures of neuromuscular work for the plurality of time points using the amplitude;
determining the plurality of muscle activation quotients, using the plurality of measures of neuromuscular work;
defining a plurality of muscle activation zones for the athlete using the muscle activation quotient, wherein individual muscle activation zones correspond to contiguous ranges of values of the muscle activation quotient;
generating a visualization of the muscle activation quotient and the plurality of muscle activation zones for the plurality of time-points; and
outputting the visualization to the display.

18. The system of claim 17, wherein the wearable muscle response sensor is a first wearable muscle response sensor, and wherein the amplitude of the muscle activity is a first amplitude of a first muscle activity of the athlete, the system further comprising a second wearable muscle response sensor configured for monitoring a second amplitude of a second muscle activity of the athlete.

19. The system of claim 18, wherein the instructions, when executed by the one or more processors, cause the one or more processors to perform further operations comprising:
monitoring the second amplitude of the second muscle of the athlete for the plurality of time points;
generating a plurality of measures of cardiorespiratory work for the plurality of time points;
determining a heart rate using the measure of cardiorespiratory work;
defining a plurality of heart rate zones using the heart rate, wherein individual heart rate zones correspond to contiguous ranges of the heart rate;
generating a second visualization of the heart rate and the plurality of heart rate zones for the plurality of time-points; and
outputting the second visualization to the display.

20. The system of claim 18, wherein the instructions, when executed by the one or more processors, cause the one or more processors to perform further operations comprising:
defining a first comparison between the plurality of muscle activation quotients and a neuromuscular work calibration curve for the athlete;
defining a second comparison between the heart rate and a cardiorespiratory calibration curve for the athlete; and
determining a fatigue status for the athlete using the comparison.
